(19) United States
(12) Patent Application Publication
(10) Pub. No.: US 2016/0287834 A1
(43) Pub. Date: Oct. 6, 2016

(54) RESUSCITATOR ARRANGEMENTS AND FLOW MONITORING

(71) Applicant: SMITHS MEDICAL INTERNATIONAL LIMITED, Ashford, kent (GB)

(72) Inventor: Paul James Leslie Bennett, Bedfordshire (GB)

(73) Assignee: SMITHS MEDICAL INTERNATIONAL LIMITED, Kent TN25 4BF (GB)

(21) Appl. No.: 14/777,764

(22) PCT Filed: Mar. 3, 2014
(86) PCT No.: PCT/GB2014/000069

§ 371 Date: Sep. 17, 2015

(30) Foreign Application Priority Data
Apr. 4, 2013 (GB) 1306067.8

(51) Int. Cl. A61M 16/20; A61M 16/10; A61M 16/06; A61M 16/04; A61M 16/08; A61M 16/00

(52) U.S. Cl. CPC A61M 16/208; A61M 16/0875; A61M 16/0057; A61M 16/06; A61M 16/04; A61M 16/1045; A61M 2205/3334

(57) ABSTRACT

A patient valve (31) for use with a resuscitator (1) has a first valve (45) opened by expiratory pressure and a second valve (71) downstream of the first that is opened by gas pressure passed by the first valve during expiration. Two pressure monitoring channels (32) and (33) extend from opposite sides of the second valve (71) to respective pressure sensors (320) and (330). A processor (340) receives the outputs of the sensors (320) and (330) to compute a differential pressure signal from which an indication of expiratory gas flow is derived. Expiratory tidal volume is derived by integrating the flow over time.

This invention relates to arrangements of the kind including a patient valve arranged to open to allow flow of gas from a gas flow conduit to a patient during an inspiratory phase and to close during an expiratory phase such that a major part of expired gas is prevented from flowing into the conduit and instead exits to atmosphere, the patient valve including a first valve element that is opened by expiratory gas pressure to allow gas to flow along an expiratory flow path resuscitator arrangements and flow monitoring.

Resuscitators are used to supply breathing gas to a patient who may not be breathing spontaneously. Portable resuscitators may take the form of a resilient bag that is squeezed manually to supply a volume of air to the patient, the bag refilling with air when it is released so that a new volume of air can be supplied. Alternatively, the resuscitator may be a mechanical device including a timing valve and various other controls and is connected to an oxygen cylinder, which both provides the breathing gas, or a part of this, and which may also provide the power to drive the components of the resuscitator. Examples of such resuscitators are described in GB 2174760, GB 2174609, EP 343818, EP 342883, EP 343824, GB 2282542, EP 691137, GB 2284159 and GB 2270629. These resuscitators are arranged to supply gas in a cyclical manner to the patient at a rate compatible with normal breathing. It is desirable to be able to provide the user of a resuscitator or ventilator with an indication of the expiratory tidal volume, that is, the volume of gas exhaled by the patient for each cycle. Conventionally flow is measured by means of a separate flow sensor connected in line between the patient valve and the patient. The flow sensor may have a flap element that moves between opposite sides of an aperture and is opened by flow in both directions but also provides an obstruction to both inspiratory and expiratory flow. Pressure lines opening on opposite sides of the flap element extend to respective pressure sensors so that an indication of the differential pressure across the flap element can be calculated and hence an indication can be derived of the flow rate. These devices have the disadvantage of obstructing both inspiratory and expiratory flow.

It is an object of the present invention to provide an arrangement and method for monitoring flow of particular use in a resuscitator or ventilator.

According to one aspect of the present invention there is provided an arrangement of the above-specified kind characterised in that the arrangement includes a second valve element located in the expiratory flow path downstream of the first valve element, that the second valve element is opened by gas pressure passed by the first valve element during an expiratory phase, that the second valve element communicates with a chamber opening to atmosphere, that the arrangement includes two pressure monitoring channels extending from the patient valve, the first channel opening to be exposed to gas pressure on an upstream side of the second valve element, the second channel opening to be exposed to gas pressure on a downstream side of the second valve element, and that the first and second channels extend to a differential pressure arrangement that provides an output indication of expiratory gas flow from the differential pressure across the second valve element.

The arrangement preferably also includes processing for deriving an indication of expiratory tidal volume from the expiratory gas flow, such as by integrating the expiratory gas flow output over time. The first and second channels preferably extend along the inside of the gas flow conduit along a major part of their length. Preferably the gas flow conduit is provided by a length of flexible corrugated tubing. The second valve element preferably includes a flap valve.

According to another aspect of the present invention there is provided a resuscitator system including a resuscitator and an arrangement according to the above one aspect of the invention where the gas flow conduit is connected to the gas outlet of the resuscitator.

The resuscitator is preferably a gas-powered resuscitator.

According to a further aspect of the present invention there is provided a method of monitoring flow in a patient breathing circuit including an expiratory flow path along which only expiratory gas flows and a one-way valve that is substantially closed during inspiratory flow and is opened during expiratory flow, characterised in that the method includes the steps of deriving a pressure output from an upstream side of the one-way valve, deriving a pressure output from a downstream side of the one-way valve, and deriving a flow indication from the upstream and downstream pressure outputs.

According to yet another aspect of the present invention there is provided a patient valve assembly including a gas flow path between a machine end and a patient end of the assembly, the gas flow path having a patient end region and a machine end region, the machine end region being arranged to receive only inspiratory gas flow supplied to the patient, the patient end region being arranged to receive both inspiratory gas flow and expiratory gas flow from the patient, characterised in that the valve assembly includes an opening from the patient end region into an expiratory gas path via a valve that is opened during an expiratory phase such that expiratory gas flows along the expiratory gas path during an expiratory phase, and that the expiratory gas path includes an arrangement located in the gas path to provide an output indication representative of gas flow along the gas path.

The arrangement located in the gas flow path preferably includes a non-return valve and first and second pressure sensing charnels opening on opposite sides of the non-return valve.

A resuscitator system including a patient breathing circuit and its method of use, according to the present invention, will now be described, by way of example, with reference to the accompanying drawings, in which:

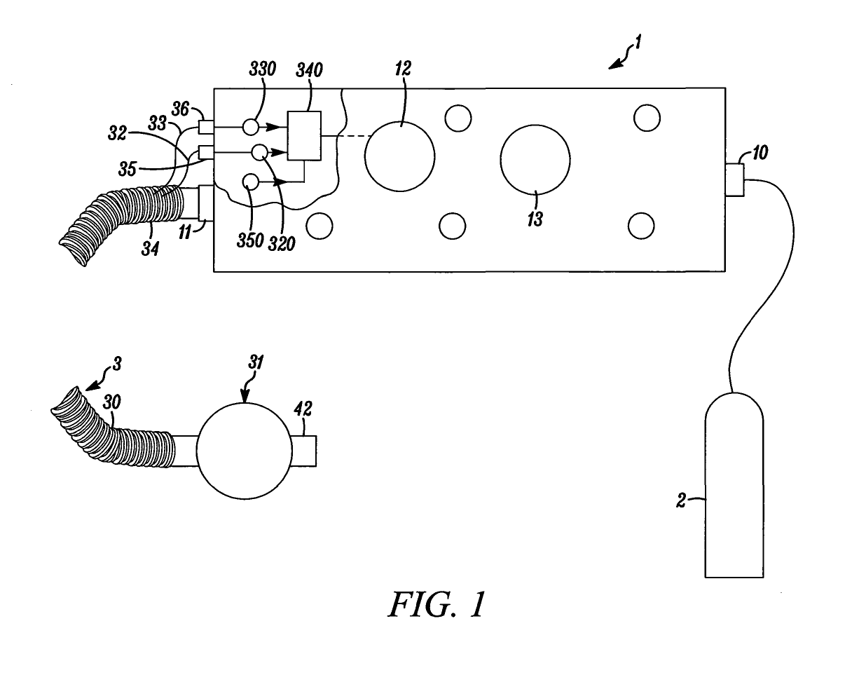

FIG. 1 shows the system schematically and illustrates schematically the pressure monitoring sensors in the resuscitator;

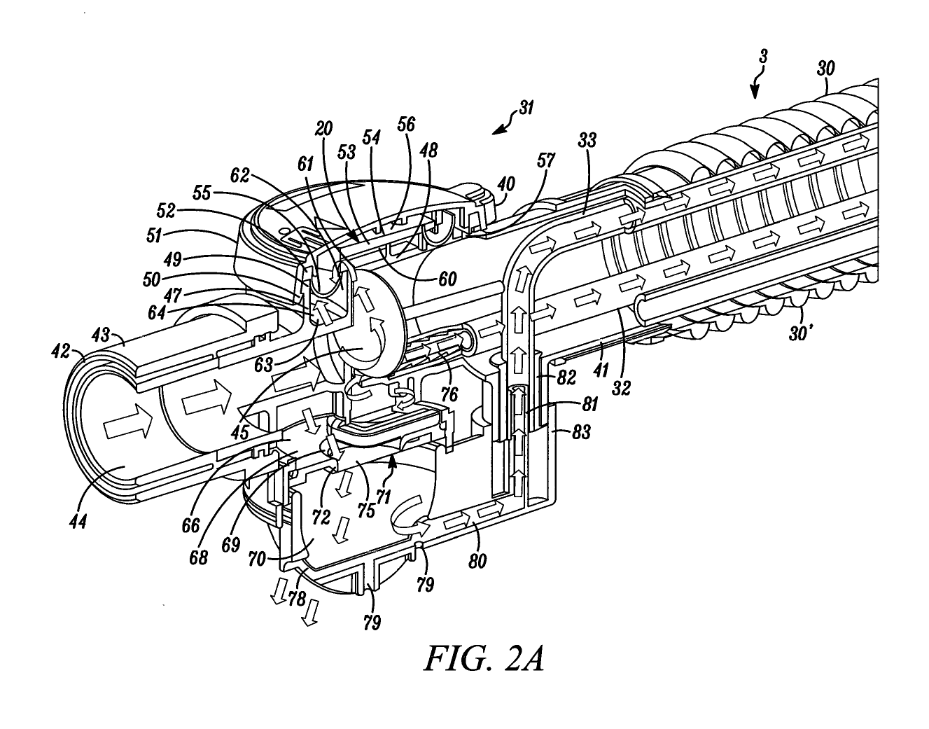

FIG. 2A is a cut-away perspective view of the patient valve assembly and the gas conduit showing gas flow during an expiratory phase;

FIG. 2B is a cut-away perspective view of the patient valve assembly and conduit during an inspiratory phase;

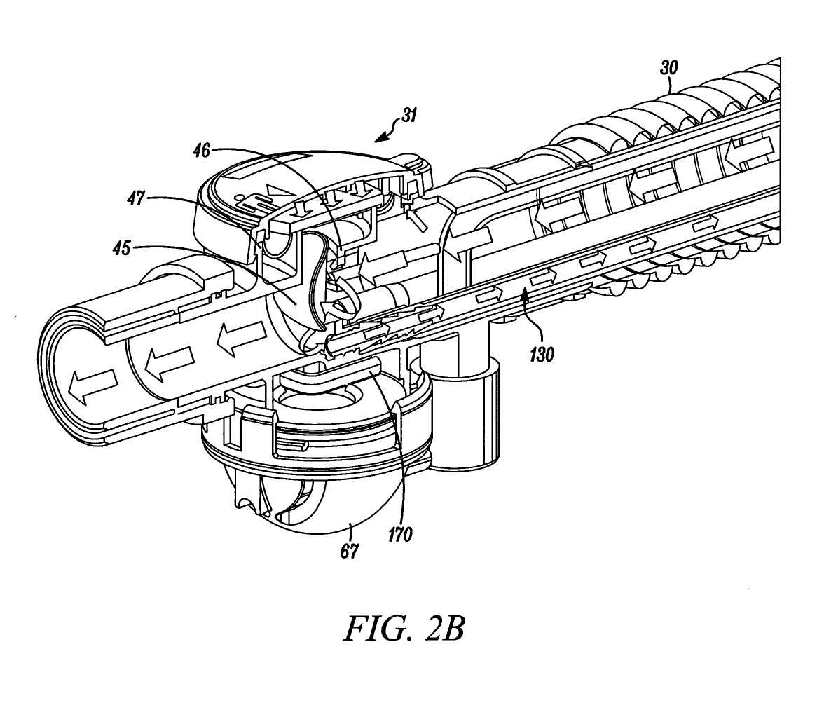

FIG. 3 is a graph illustrating how differential pressure changes with inspiratory and expiratory flow;

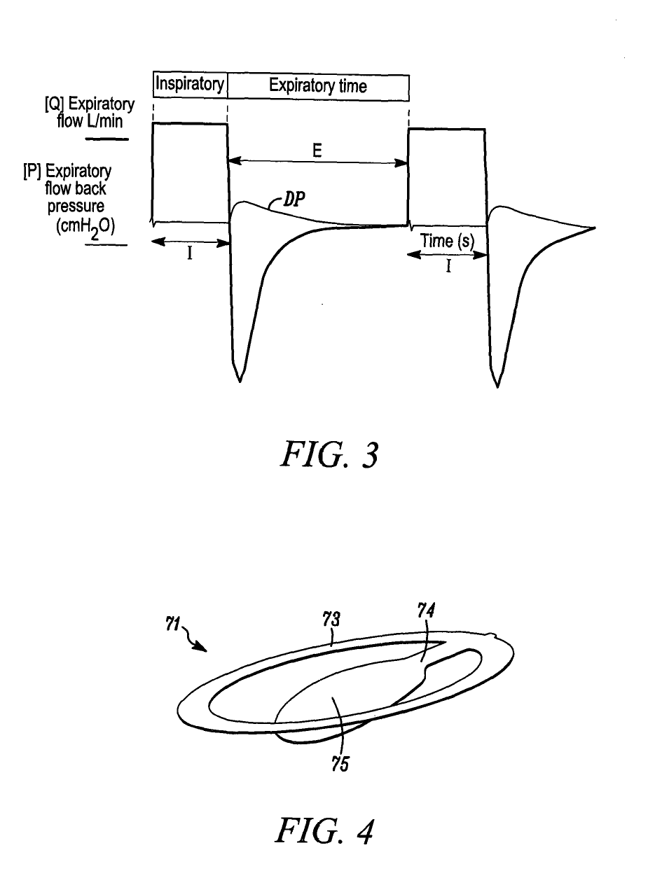

FIG. 4 is a perspective view of the non-return flap valve in the outlet of the patient valve.

With reference first to FIG. 1, the resuscitator system comprises a resuscitator unit 1, an oxygen cylinder 2 connected to a gas supply inlet 10 on the resuscitator, and a patient breathing circuit 3 connected to a ventilation gas outlet 11.

The resuscitator unit 1 is similar to that sold by Smiths Medical International Limited under the Parapac trade mark (Parapac is a Registered Trade Mark of Smiths Medical International Limited) with the addition of a function for calculating and indicating expiratory tidal volume on a display 12. The resuscitator unit 1 is of the mechanical, gas-powered kind where the gas from the oxygen cylinder 2 provides both the breathing gas, or a part of the breathing gas, and also provides the power to drive the components of the resuscitator such as the timing valve, air entrainment mixer and various other controls. The resuscitator 1 provides an adjustable, cyclical gas mixture during inspiratory breathing phases to the gas outlet 11, and from there to the breathing circuit 3.

The breathing circuit 3 includes two main components, namely a gas supply conduit 30 and a patient valve assembly 31. The gas supply conduit 30 takes the form of a flexible, corrugated tube 30′ and also includes two small-bore pressure monitoring channels or lines 32 and 33 extending along the interior of the tube. At the machine end 34 of the breathing circuit 3 the pressure lines 32 and 33 connect with respective spigots 35 and 36, which make gas connection with respective pressure sensors 320 and 330 in the resuscitator 1 the function of which will be described later. The spigots 35 and 36 may be located within the gas outlet 11 or to one side of it.

With reference to FIGS. 2A and 2B, the patient valve 31 includes an outer housing 40 of generally T shape in section having a tubular machine end extension 41 at one end onto which the patient end of the conduit 30 is fitted. At the opposite end of the housing 40 a patient end extension 42 extends forwardly in axial alignment with the machine end extension 41. The patient end extension 42 has a conventional luer tapered coupling surface 43. In use, the patient end extension 42 would be connected to a face mask, a tracheal tube, laryngeal mask or other airway by which gas can be supplied to and from the patient. Usually, an HME (heat and moisture exchange element) would be connected between the patient valve 31 and the face mask or the like. This helps conserve warmth and moisture within the patient's breathing passages by exchanging exhaled heat and moisture to inhaled gas. It also helps reduce the amount of moisture entering the patient valve 31 and so reduces the build up of condensation within it.

A through bore 44 extends along the housing 40 between the patient and machine end extensions 42 and 41. A flexible flap valve 45 of circular shape is mounted midway along the through bore 44 extending laterally of the bore and with its rear, machine side bearing on an annular valve seat 46 (shown more clearly in FIG. 2B) formed around the inside of the housing 40. As shown in FIG. 2B, the flap valve 45 flexes away from the valve seat 46 during the inspiratory phase, when gas pressure on the machine side of the valve exceeds that on the opposite side, to allow gas to flow along the bore through the patient valve 31 between opposite ends. Pressure on the patient side of the flap valve 45 is communicated to a pressure gauge 13 in the resuscitator unit 1 via a small-bore tube 130 extending along the conduit 30. This pressure is preferably also supplied to an alarm to provide a warning when pressure is outside predetermined limits. During the expiratory phase (FIG. 2A) the gas pressure on the patient side of the flap valve 45 exceeds that on its opposite side so that it is forced against the valve seat 46, thereby preventing gas flowing past the flap valve.

An exhalation valve 20 is located in the valve housing 40 above the flap valve 45. This region the valve housing 40 is formed with an upwardly-projecting circular rim 47 around a base plate 48 extending in a plane parallel to the axis of the through bore 44. The rim 47 has a tapered lip 49 around its outside to which a corresponding lip 50 around the inside of a circular exhalation valve cap 51 is clipped to retain the cap in position on the housing 40. The cap 51 traps the outer edge 52 of an exhalation valve element 53 extending across the base plate 48. The exhalation valve element 53 is in the form of a circular resilient membrane having a flat central region 54 and a peripheral U-shape edge region 55 that is thinner and more flexible than the central region. The cap 51 defines a small volume upper chamber 56 above the valve element 53 that communicates with the bore 44 on the machine side of the flap valve 45 via a first, small opening 57 in the base plate 48 just outside the edge 52 of the valve element 53. A similar small volume lower chamber 60 is defined below the valve element 53 above the base plate 48. A second small opening 61 in the base plate 48 opens into the lower chamber 60 from the through bore 44 on the patient side of the flap valve 45.

The underside of the exhalation valve element 53 normally sits against a ledge 62 projecting from the base plate 48 at an outer edge of the lower chamber 60 to block escape of gas from the lower chamber. When the valve element 53 is lifted by a change in differential pressure across the element a gap is formed between the upper edge of the ledge 62 and the underside of the valve element opening into one end of an expiratory gas flow path 63. The gas flow path 63 is formed by a horizontal channel 64 extending laterally under the patient end side of the U-shape edge region 55 that opens into two vertical channels (not visible in the drawings) extending down opposite sides of the housing 40.

The vertical channels open into an upstream vent chamber 66 formed at the upper end of a domed outlet moulding 67 depending from the lower side of the patient valve assembly 31. The upstream chamber 66 has a floor 68 with a hole 69 through it opening into a lower, downstream vent chamber 70. The lower side of the hole 69 is covered by a non-return valve 71 that bears on a downwardly-projecting lip 72 around the hole forming a valve seat. The non-return valve 71 is, therefore, located downstream of the exhalation valve 20. The non-return valve 71 is shown most clearly in FIG. 4 comprising an outer circular mounting ring 73 from which a short tongue 74 projects radially inwardly, supporting at its inner end a circular valve plate element 75 located concentrically within the ring 73. The non-return valve 71 is an integral, unitary, one-piece component stamped from a thin sheet of polyester material (such as Melinex 401CW). The valve 71 is attached to the under surface of the floor 68 by the mounting ring 73, with the plate element 75 positioned over the hcle 69. The construction and nature of the material of the non-return valve 71 is such that the plate element 75 only makes light contact with the valve seat 72 and readily opens when pressure in the upstream vent chamber 66 rises slightly. In its natural state, the plate element 75 may not, in fact, contact the valve seat 72 but, if a negative pressure were applied above the valve, such as caused by the patient inhaling, this would draw the plate element into sealing contact with the valve seat. One function of the non-return valve 71 is, therefore, to ensure that, if the patient inhales, gas is inhaled from the resuscitator and not via the expiratory gas flow path. The upstream vent chamber 66 also communicates with one of the pressure lines 32, which is fitted onto the outside of one end of a barbed spigot 76, the other end of which opens into the upstream vent chamber. In this way, pressure changes in the upstream vent chamber 66 are communicated to the pressure sensor 320 in the resuscitator 1. A partition plate 170 acts as a tray to collect condensation in the region of the open end of the pressure monitoring line 32 so that it can run away from the pressure line.

The downstream vent chamber 70 has an outlet aperture 78 opening to atmosphere and has two condensation drain holes 79 at its lower end to allow any condensation that might collect in the chamber to drain out. The chamber 70 also has a downstream pressure monitoring channel 80 communicating with a vertical tapered spigot 81. The patient end of the second pressure monitoring line 33 is fitted over the upper end of the spigot 81 and is held securely in place by a sleeve 82 pushed down around the line over the spigot and within a tubular side port 83 projecting from the patient end of the conduit 30.

The two pressure signals supplied along lines 32 and 33 are, therefore, derived from opposite sides of the non-return valve 71, which provides an obstruction or bluff body in the expiratory flow path 63 only.

In operation, during the inspiratory phase, the resuscitator unit 1 provides a positive gas pressure to its outlet 11 and hence to the machine end of the conduit 30. This gas pressure is communicated to the patient end of the conduit 30 and to the patient valve assembly 31. The positive pressure on the machine side of the flap valve 45 causes this to open so that gas can flow past the valve to the patient end 42 of the patient valve 31 and hence to the patient. Positive pressure on the machine end side of the flap valve 45 also causes gas to flow through the opening 57 to the upper chamber 56 above the exhalation valve element 53 ensuring that this is held down against the ledge 62 and closes the flow path via the other opening 61 to ensure that gas does not escape via the expiratory flow path 63. This phase is indicated by the letter “I” in the graph of FIG. 3. Positive flow in this graph represents flow to the patient; negative flow represents flow from the patient. It can be seen that during the inspiratory phase “I” no gas is supplied to the upstream and downstream vent chambers 66 and 70 so the pressure on both sides of the non-return flap valve 71 is equal, giving a zero differential pressure output and hence a zero flow output.

During the expiratory phase “E”, the resuscitator unit 1 stops supplying gas to the conduit 30 thereby allowing the patient to exhale and supply an expiratory gas stream to the patient end 42 of the patient valve assembly 31. This increases pressure on the patient side of the flap valve 45 forcing its opposite side into contact with the valve seat 46. Expiratory gas flows through the opening 61 into the lower chamber 60 beneath the expiratory valve element 53, thereby lifting the valve element and allowing gas to flow over the ledge 62 and into the expiratory gas flow path 63. Pressure, therefore, increases in the upstream vent chamber 66, opening the non-return valve 71 and allowing gas to flow into the downstream vent chamber 70 and to atmosphere via the outlet 78. This causes the pressure signals from opposite sides of the non-return valve 71 to be communicated along the pressure lines 32 and 33 to the sensors 320 and 330 in the resuscitator. The pressure in the upstream chamber 66 is higher than that in the downstream chamber 70 leading to a positive differential pressure as indicated by the line “DP” in FIG. 3. This differential pressure is measured by a processor 340 in the resuscitator 1 and is converted to a flow rate measurement. The flow measurement is then converted to a total expiratory tidal volume measurement by integrating over the expiratory period and this is indicated on the display 12. The resuscitator 1 also includes a barometric pressure sensor 350 exposed to atmospheric pressure and connected with the processor 340 so that an appropriate correction can be made for changes in atmospheric pressure.

The small diameter of the pressure lines 32 and 33 makes them prone to blockage by particles, secretions or fluids (especially if these freeze during cold conditions). In order to prevent this, the resuscitator is arranged periodically automatically to supply a short positive pulse of gas to both lines such as once per inspiratory phase, or once every few inspiratory phases.

The present invention enables an indication of expiratory tidal volume to be provided without the need to obstruct the inspiratory gas flow path. Also, by incorporating the flow sensing within the patient valve itself it leads to a more compact configuration and avoids the need to connect separate components to the patient valve.

1-11. (canceled)

12. An arrangement including a patient valve arranged to open to allow flow of gas from a gas flow conduit to a patient during an inspiratory phase and to close during an expiratory phase such that a major part of expired gas is prevented from flowing into the conduit and instead exits to atmosphere, the patient valve including a first valve element that is opened by expiratory gas pressure to allow gas to flow along an expiratory flow path, characterized in that the arrangement includes a second valve element located in the expiratory flow path downstream of the first valve element, that the second valve element is opened by gas pressure passed by the first valve element during an expiratory phase, that the second valve element communicates with a chamber opening to atmosphere, that the arrangement includes two pressure monitoring channels extending from the patient valve, the first channel opening to be exposed to gas pressure on an upstream side of the second valve element, the second channel opening to be exposed to gas pressure on a downstream side of the second valve element, and that the first and second channels extend to a differential pressure arrangement that provides an output indication of expiratory gas flow from the differential pressure across the second valve element.

13. An arrangement according to claim 12, characterized in that the arrangement includes a processor for deriving an indication of expiratory tidal volume from the expiratory gas flow.

14. An arrangement according to claim 13, characterized in that the indication of expiratory tidal volume is derived by integrating the expiratory gas flow output over time.

15. An arrangement according to claim 12, characterized in that the first and second channels extend along the inside of the gas flow conduit along a major part of their length.

16. An arrangement according to claim 12, characterized in that the gas flow conduit is provided by a length of flexible corrugated tubing.

17. An arrangement according to claim 12, characterized in that the second valve element includes a flap valve.

18. A resuscitator system including a resuscitator and an arrangement including a patient valve arranged to open to allow flow of gas from a gas flow conduit to a patient during an inspiratory phase and to close during an expiratory phase such that a major part of expired gas is prevented from flowing into the conduit and instead exits to atmosphere, the patient valve including a first valve element that is opened by expiratory gas pressure to allow gas to flow along an expiratory flow path, characterized in that the arrangement includes a second valve element located in the expiratory flow path downstream of the first valve element, that the second valve element is opened by gas pressure passed by the first valve element during an expiratory phase, that the second valve element communicates with a chamber opening to atmosphere, that the arrangement includes two pressure monitoring channels extending from the patient valve, the first channel opening to be exposed to gas pressure on an upstream side of the second valve element, the second channel opening to be exposed to gas pressure on a downstream side of the second valve element, and that the first and second channels extend to a differential pressure arrangement that provides an output indication of expiratory gas flow from the differential pressure across the second valve element; wherein the gas flow conduit is connected to the gas outlet of the resuscitator.

19. A resuscitator according to claim 18, characterized in that the resuscitator is a gas-powered resuscitator.

20. A resuscitator according to claim 18, characterized in that the arrangement includes a processor for deriving an indication of expiratory tidal volume from the expiratory gas flow.

21. A resuscitator according to claim 20, characterized in that the indication of expiratory tidal volume is derived by integrating the expiratory gas flow output over time.

22. A resuscitator according to claim 18, characterized in that the first and second channels extend along the inside of the gas flow conduit along a major part of their length.

23. A resuscitator according to claim 18, characterized in that the gas flow conduit is provided by a length of flexible corrugated tubing.

24. A resuscitator according to claim 18, characterized in that the second valve element includes a flap valve.

25. A method of monitoring flow in a patient breathing circuit including an expiratory flow path along which only expiratory gas flows and a one-way valve that is substantially closed during inspiratory flow and is opened during expiratory flow, characterized in that the method includes the steps of deriving a pressure output from an upstream side of the one-way valve, deriving a pressure output from a downstream side of the one-way valve, and deriving a flow indication from the upstream and downstream pressure outputs.

26. A patient valve assembly including a gas flow path between a machine end and a patient end of the assembly, the gas flow path having a patient end region and a machine end region, the machine end region being arranged to receive only inspiratory gas flow supplied to the patient, the patient end region being arranged to receive both inspiratory gas flow and expiratory gas flow from the patient, characterized in that the valve assembly includes an opening from the patient end region into an expiratory gas path via a valve that is opened during an expiratory phase such that expiratory gas flows along the expiratory gas path during an expiratory phase, and that the expiratory gas path includes an arrangement located in the gas path to provide an output indication representative of gas flow along the gas path.

27. A patient valve assembly according to claim 26, characterized in that the arrangement located in the gas flow path includes a non-return valve and first and second pressure sensing channels opening on opposite sides of the non-return valve.
